(19) United States
(12) Patent Application Publication
(10) Pub. No.: US 2022/0084669 A1
(43) Pub. Date: Mar. 17, 2022

(54) METHOD FOR PERFORMING COMPLEX COMPUTING ON VERY LARGE SETS OF PATIENT DATA

(71) Applicant: KONINKLIJKE PHILIPS N.V., EINDHOVEN (NL)

(72) Inventors: Joep Joseph Benjamin Nathan VAN BERKEL, Gronsveld (NL); Jan Johannes Gerardus DE VRIES, Leende (NL)

(21) Appl. No.: 17/420,405

(22) PCT Filed: Dec. 20, 2019
(86) PCT No.: PCT/EP2019/086502

§ 371 Date: Jul. 2, 2021

Related U.S. Application Data
(60) Provisional application No. 62/787,921, filed on Jan. 3, 2019.

(51) Int. Cl. G16H 40/63; G16H 10/60

(52) U.S. Cl. CPC G16H 40/63; G16H 10/60

(57) ABSTRACT

A method for generating virtual patients, including: collecting patient data including features for a plurality of patients; clustering the plurality of patients based upon the features to define patient data sub-groups in the plurality of patients; determining the homogeneity of the patient data sub-groups; and generating virtual patients for each patient data sub-group that represent the features of the patient data sub-group.

### TECHNICAL FIELD

Various exemplary embodiments disclosed herein relate generally to a method for performing complex computing on very large sets of patient data.

### BACKGROUND

Hospitals are in a continuous effort to optimize their care, lower cost, and improve the experience of care for their patient population. In these attempts, data analysis plays a key role to identify gaps in care, areas of improvement and underperformance, and optimal care provision to their patient base. As the amount and diversity and availability of multisource data increases, health data analytics solutions are enabling extraction of actionable and meaningful insights from these data to support optimization of mentioned care provision and improvement of outcomes.

In order to utilize these large amounts of patient data, more and more complex computations are performed on this patient data. With the increasing amount of data and patients in a care population, the time and computational power to perform these calculations grows rapidly.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for generating virtual patients, including: collecting patient data including features for a plurality of patients; clustering the plurality of patients based upon the features to define patient data sub-groups in the plurality of patients;

determining the homogeneity of the patient data sub-groups; and generating virtual patients for each patient data sub-group that represent the features of the patient data sub-group.

Various embodiments are described, wherein generating the virtual patient includes selecting an actual patient based upon the mode of the patient data for the patient data sub-group.

Various embodiments are described, wherein generating the virtual patient includes defining the features of the virtual patient based upon the average of the patient data for the patient data sub-group.

Various embodiments are described, wherein generating the virtual patient includes defining the features of the virtual patient based upon the median of the patient data for the patient data sub-group.

Various embodiments are described, further including clustering a sub-group when the homogeneity of the patient data sub-group is below a specified value.

Various embodiments are described, further including: determining care plans associated with each virtual patient; selecting a patient population; adding the virtual patients to the patient population; clustering the patient population with the virtual patient to define patient sub-groups in the patient population; identifying the virtual patients in each patient sub-group; and selecting a care plan for each patient in the patient sub-group based upon the virtual patient in the patient sub-group.

Various embodiments are described, wherein selecting a care plan for each patient in the patient sub-group is further based upon patient one of patient inclusion criteria and patient eligibility criteria.

Various embodiments are described, further including determining the inclusion criterial for each care plan associated with each virtual patient.

Various embodiments are described, further including: determining care plans associated with each virtual patient; selecting a patient population; clustering the patient population to define patient sub-groups in the patient population; adding the virtual patients to the nearest patient sub-group of the patient population; and selecting a care plan for each patient in the patient sub-group based upon the virtual patient associated with the patient sub-group.

Various embodiments are described, wherein selecting a care plan for each patient in the patient sub-group is further based upon one of patient inclusion criteria and patient eligibility criteria.

Various embodiments are described, further including determining the inclusion criterial for each care plan associated with each virtual patient.

Various embodiments are described, further including: determining care plans associated with each virtual patient; selecting a patient population; mapping the virtual patients into the patient population space; determine which patients are within a certain distance of each virtual patient; and selecting a care plan for each patient based upon the virtual patient associated with each patient.

Various embodiments are described, wherein selecting a care plan for each patient is further based upon one of patient inclusion criteria and patient eligibility criteria.

Various embodiments are described, further including determining the inclusion criterial for each care plan associated with each virtual patient.

Various embodiments are described, wherein selecting a care plan for each patient further includes, when a patient is within the certain distance of two virtual patients, selecting the care plan associated with the virtual patient closest to the patient.

Further various embodiments relate to a non-transitory machine-readable storage medium encoded with instructions for generating virtual patients, including: instructions for collecting patient data including features for a plurality of patients; instructions for clustering the plurality of patients based upon the features to define patient data sub-groups in the plurality of patients; instructions for determining the homogeneity of the patient data sub-groups; and instructions for generating virtual patients for each patient data sub-group that represent the features of the patient data sub-group.

Various embodiments are described, wherein instructions for generating the virtual patient includes instructions for selecting an actual patient based upon the mode of the patient data for the patient data sub-group.

Various embodiments are described, wherein instructions for generating the virtual patient includes instructions for defining the features of the virtual patient based upon the average of the patient data for the patient data sub-group.

Various embodiments are described, wherein instructions for generating the virtual patient includes instructions for defining the features of the virtual patient based upon the median of the patient data for the patient data sub-group.

Various embodiments are described, further including instructions for clustering a sub-group when the homogeneity of the patient data sub-group is below a specified value.

Various embodiments are described, further including: instructions for determining care plans associated with each virtual patient; instructions for selecting a patient population; instructions for adding the virtual patients to the patient population; instructions for clustering the patient population with the virtual patient to define patient sub-groups in the patient population;

instructions for identifying the virtual patients in each patient sub-group; and instructions for selecting a care plan for each patient in the patient sub-group based upon the virtual patient in the patient sub-group.

Various embodiments are described, wherein instructions for selecting a care plan for each patient in the patient sub-group is further based upon patient one of patient inclusion criteria and patient eligibility criteria.

Various embodiments are described, further including instructions for determining the inclusion criterial for each care plan associated with each virtual patient.

Various embodiments are described, further including: instructions for determining care plans associated with each virtual patient; instructions for selecting a patient population; instructions for clustering the patient population to define patient sub-groups in the patient population; instructions for adding the virtual patients to the nearest patient sub-group of the patient population; and instructions for selecting a care plan for each patient in the patient sub-group based upon the virtual patient associated with the patient sub-group.

Various embodiments are described, wherein instructions for selecting a care plan for each patient in the patient sub-group is further based upon one of patient inclusion criteria and patient eligibility criteria.

Various embodiments are described, further including instructions for determining the inclusion criterial for each care plan associated with each virtual patient.

Various embodiments are described, further including: instructions for determining care plans associated with each virtual patient; instructions for selecting a patient population; instructions for mapping the virtual patients into the patient population space; instructions for determine which patients are within a certain distance of each virtual patient; and instructions for selecting a care plan for each patient based upon the virtual patient associated with each patient.

Various embodiments are described, wherein instructions for selecting a care plan for each patient is further based upon one of patient inclusion criteria and patient eligibility criteria.

Various embodiments are described, further including instructions for determining the inclusion criterial for each care plan associated with each virtual patient.

Various embodiments are described, wherein instructions for selecting a care plan for each patient further includes, when a patient is within the certain distance of two virtual patients, instructions for selecting the care plan associated with the virtual patient closest to the patient.

### BRIEF DESCRIPTION OF THE DRAWINGS

In order to better understand various exemplary embodiments, reference is made to the accompanying drawings, wherein:

FIG. 1. illustrates a method for generating virtual subjects by the data processing system;

FIG. 2 illustrates first a care plan assignment method;

FIG. 3 illustrates the application of the method for generating virtual subjects to patient data to determine a set of virtual patients;

FIG. 4 provides an illustration of the application of the care plan assignment method of FIG. 2 to a patient population;

FIG. 5 illustrates second a care plan assignment method;

FIG. 6 provides an illustration of the application of the care plan assignment method of FIG. 5 to a patient population;

FIG. 7 illustrates third a care plan assignment method; and

FIG. 8 provides an illustration of the application of the care plan assignment method of FIG. 7 to a patient population.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

A data processing system described herein allows for what may be called compression of the population data such that computations may be performed on a representative subset of data points, however the computations still reflect the total patient population. This allows for various computations to be performed on the patient population data by the data processing system in less time. It allows for the computations to be performed more often.

As an example embodiment of the data processing system, care plan assignment and the optimization thereof will be described. This requires the comparison of individual patients against large reference populations. This example embodiment of the data processing systems aims to support users /care managers in matching individuals to care plans benefit those individuals the most based on defining virtual patients. These virtual patients may be defined by features extracted from data of similar patients that completed their care plans and for whom outcomes are available. The virtual patients may then be injected into the clustering results of active patients. The care plans and outcomes linked to the virtual patients would then be evaluated, and the active patients would then be matched to the identified, most optimal care plans. The results of the analysis are then to be confirmed by a care manager.

The data processing system implementing a care plan selection method aims to represent a patient population by means of a smaller set of representative or virtual patients. The data processing system uses as input a variety of data on clinical, medical, claims, demographic, social determinants of health, and utilization features of the patient population originating, for example, from the electronic medical records (EMR), claims data, and potentially other data sources (e.g., claims-based systems, lab-systems, socio-economic sources, etc.).

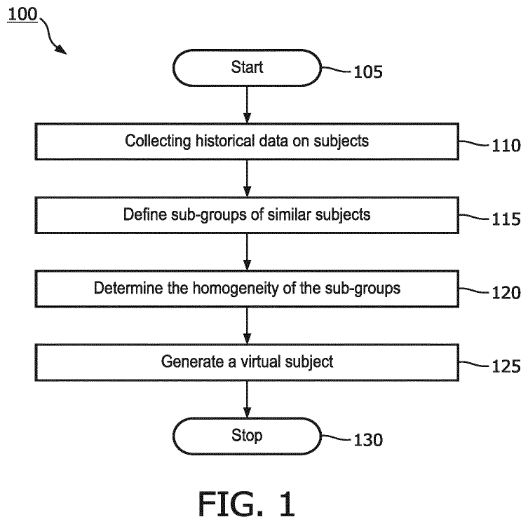

FIG. 1. illustrates a method 100 for generating virtual subjects by the data processing system. The data processing system first collects the patient data (from different sources) on subjects from a given population 110. This yields a set of feature values for each subject. In the embodiments described herein the subjects are patients, but could be other items as well where the subject need to be matched to another subject associated with an action plan.

Next, the data processing system 100, defines sub-groups of similar patients from step 110 by clustering these subject along a selection of features related to the subject. If the subjects are patients, the selection may include the clinical, medical, claims, demographic, socioeconomic and utilization features of these patients. As the goal is to define virtual subjects, the sub-groups resulting from the clustering technique should have a homogeneity level above a desired threshold level. Clustering may be performed by an existing clustering method such as agglomerative hierarchical clustering (AHC), K-means, density-based spatial clustering of applications with noise (DBSCAN), balanced iterative reducing and clustering using hierarchies (BIRCH), etc. For the embodiments described herein, AHC is used. In its simplest form, the clustering algorithm is applied once to form clusters that will be evaluated in the next step, but an alternative embodiment could allow for the reapplication of the clustering technique on clusters that do not meet the threshold for homogeneity of the composition of the cluster to form smaller and more homogeneous clusters. The clustering technique will group together subjects that are similar in terms of the input data that characterizes the subjects and form distinct clusters that show more differences between clusters than within clusters.

The data processing system then determines the homogeneity of the sub-groups 120. Sub-groups with a homogeneity below a pre-set threshold may be re-clustered by applying the clustering technique from 115 on the subset of subjects in this cluster. To define homogeneity of the sub-groups various methods may be applied such as the silhouette coefficient, Davies-Bouldin index, Dunn, etc.

Finally, the data processing system generates a virtual subject 125. The virtual subject is a representation of the patients that make up the sub-group. Each sub-group would thus be represented by a virtual subject. Some sub-groups could potentially be represented by multiple virtual subjects, if the sub-group is not very homogeneous. For all sub-groups, it holds that if homogeneity of a sub-group is above a certain threshold the features of these patients are combined to form a virtual patient (i.e., a medoid representation) of the sub-group. Note that there are various techniques to come to such a medoid representation. Depending on the exact application it could be preferred to select an actual patient to form the medoid representation (e.g., by selecting the mode of the data in the sub-group), or by applying some function like the average or median on the data from the sub-group. Some sub-groups could potentially be represented by multiple virtual patients if the sub-group is not very homogeneous. The method 100 then ends at 130.

Now an embodiment of the data processing system will be described that helps to optimize the selection of care plans for patients. But other embodiments are contemplated where one would want to allow for what could be called compression of the population data, such that the computations may be performed on a representative subset of data points, but still reflect the total patient population.

To this end, the method 100 is applied to patient data including the various sources such as the clinical, medical, claims, demographic, socioeconomic and utilization features of these patients (e.g., the various sources available in the EMR), and also indicators for each patient of whether they are enrolled in a care plan as well as the patient's medical outcomes. Now, for unseen patients, the goal is to find the best set of care plans for that patient. To that end, each patient is to be compared against the population, but rather than comparing against all patients, the unseen patient is compared against the set of virtual patients representing entire sub-groups of similar patients.

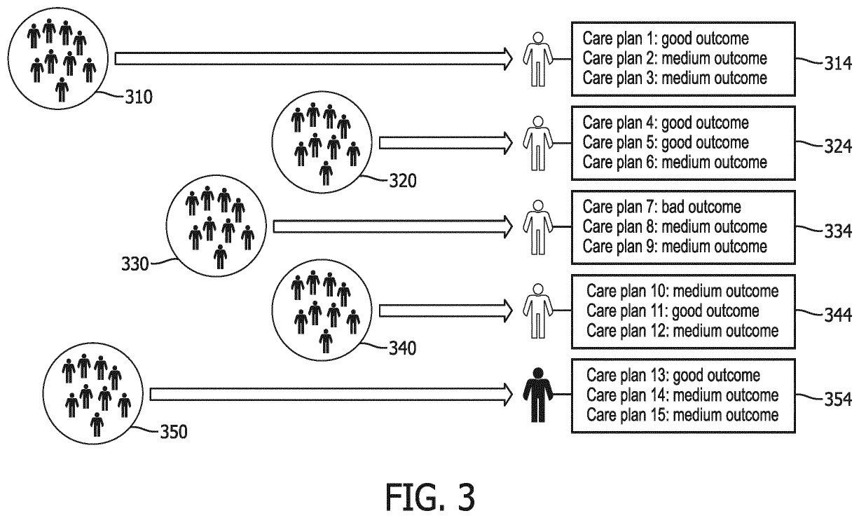

FIG. 3 illustrates the application of the method 100 to patient data to determine a set of virtual patients. The method 100 clusters the patient population into a set of clusters 310, 320, 330, 340, and 350. The method generates a virtual patients 312, 322, 332, 342, and 352 for each of the clusters. Then for each virtual patient that describes a sub-group an inventory 314, 324, 334, 344, and 354 is made of which care plans are assigned to the patients in that sub-group that contributed to the make-up of the virtual patients, as well as inclusion criteria and outcomes related to the identified care plans in the sub-groups are identified. This results in a list of one or more virtual patients for each sub-group, where each virtual patient is linked to a list of care plans, related inclusion criteria, and related outcomes the patients contributing to defining this virtual patient had been assigned to.

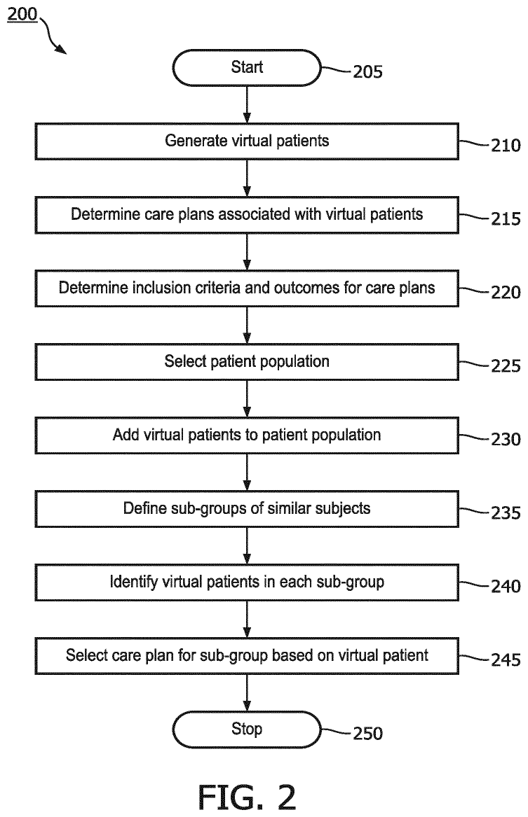

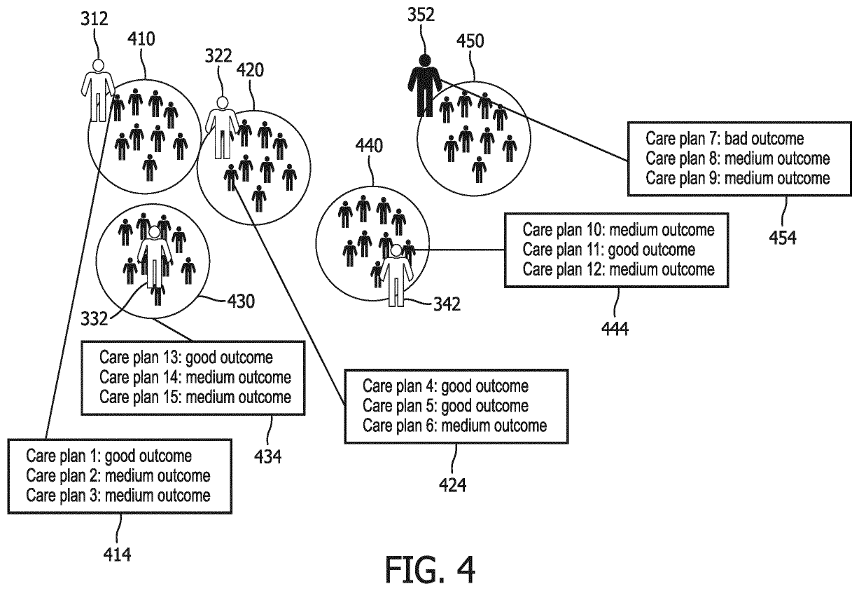

FIG. 2 illustrates first a care plan assignment method 200. FIG. 4 provides an illustration of the application of the care plan assignment method 200 of FIG. 2 to a patient population. The care plan assignment method 200, for each unseen patient, selects the most similar virtual patient based upon the following steps. The care plan assignment method 200 starts at 205 and then generates virtual patients 210 as described above using the method 100. The care plan assignment method 200 then determines the care plans associated with the virtual patients 215 as well as the inclusion criteria and outcomes for the care plans 220 as shown in FIG. 3. The care plan assignment method 200 then selects a patient population 225. This may be accomplished via input from a user of the system or automatically performed. Next, the care plan assignment method 200 adds the virtual patients to the patient population 230. Then the care plan assignment method 200 clusters the patient population 235 into sub-groups including the virtual patients using methods like those described above in step 115. This is shown in FIG. 4 where clusters 410, 420, 430, 440, and 450 have been formed. The care plan assignment method 200 then identifies the virtual patient(s) in each sub-group 240. This is shown in FIG. 4 where virtual patients 312, 322, 332, 342, and 352 have been assigned to clusters 410, 420, 430, 440, and 450 respectively. Then the care plan assignment method 200 selects a care plan for each sub-group based on the associated virtual patient 245. This is shown in FIG. 4 where the list of care plans 414, 424, 434, 444, and 454 associated with the virtual patients 312, 322, 332, 342, and 352 are shown. The best care plan in the list of care plans will be selected for each patient in the sub-group subject to inclusion and eligibility criteria for the care plans. In some situations, multiple virtual patients may be associated with a sub-group. In that case, the best care plan from among the virtual patients may be selected. Alternatively, the closest virtual patient to each patient in the sub-group may be determined, and the best care plan selected based upon the closest virtual patient. As a result, patients in the same sub-group may have different care plans assigned because of inclusion and eligibility criteria or because of multiple virtual patients being associated with the sub-group or both. The method then ends at 250. An effect of this care plan assignment method 200 is that the virtual patients would influence the generation and content of the sub-groups; but this influence may actually be beneficial in generating sub-groups relevant to the care plans and interventions linked to the virtual patient.

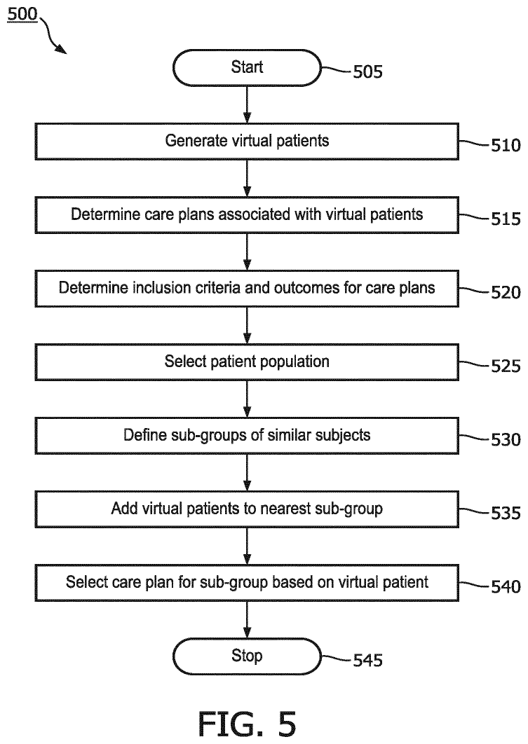

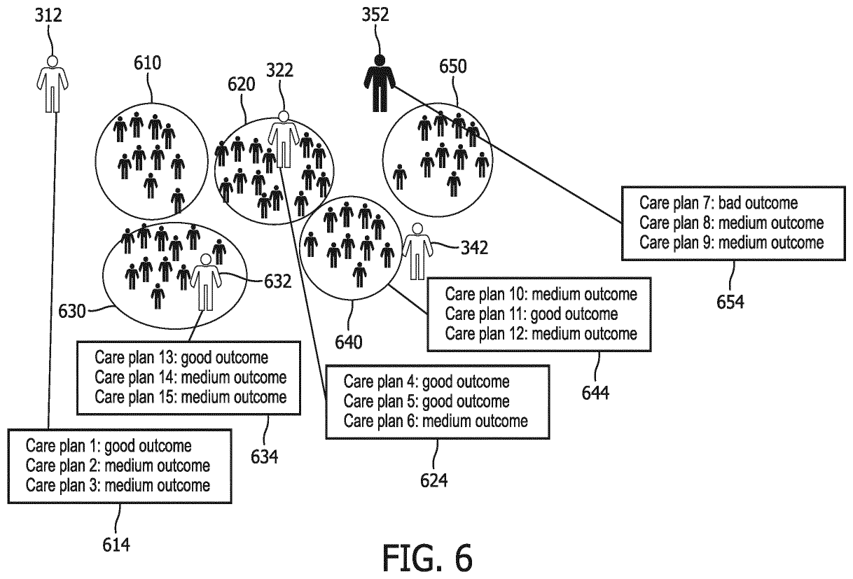

FIG. 5 illustrates second a care plan assignment method 500. FIG. 6 provides an illustration of the application of the care plan assignment method 500 of FIG. 5 to a patient population. The care plan assignment method 500, for each unseen patient, selects the most similar virtual patient based upon the following steps. The care plan assignment method 500 starts at 505 and then generates virtual patients 510 as described above using the method 100. The care plan assignment method 500 then determines the care plans associated with the virtual patients 515 as well as the inclusion criteria and outcomes for the care plans 520 as shown in FIG. 3. The care plan assignment method 500 then selects a patient population 525. This may be accomplished via input from a user of the system or automatically performed. Then the care plan assignment method 500 clusters the patient population 530 using methods like those described above in step 115. This is shown in FIG. 6 where clusters 610, 620, 630, 640, and 650 have been formed. The care plan assignment method 500 then adds the virtual patients the nearest sub-group 535 with whom they show a high degree of similarity with. This may be done using the shortest distance to the centroid or any other cluster parameter of the cluster as defined by the distance measure used for clustering. Then a similarity above a certain threshold is indicated by a distance shorter than a certain threshold. This is shown in FIG. 6 where virtual patients 312, 322, 332, 342, and 352 have been assigned to clusters 610, 620, 630, 640, and 650 respectively. Then the care plan assignment method 500 selects a care plan for each sub-group based on the associated virtual patient 540. This is shown in FIG. 6 where the list of care plans 614, 624, 634, 644, and 654 associated with the virtual patients 312, 322, 332, 342, and 352 are shown. The best care plan in the list of care plans will be selected for each patient in the sub-group subject to inclusion and eligibility criteria for the care plans. In some situations, multiple virtual patients may be associated with a sub-group. In that case, the best care plan from among the virtual patients may be selected. Alternatively, the closest virtual patient to each patient in the sub-group may be determined, and the best care plan selected based upon the closest virtual patient. As a result, patients in the same sub-group may have different care plans assigned because of inclusion and eligibility criteria or because of multiple virtual patients being associated with the sub-group or both. The method then ends at 545. An effect of this care plan assignment method 500 is that the sub-groups are generated independent of the virtual patients.

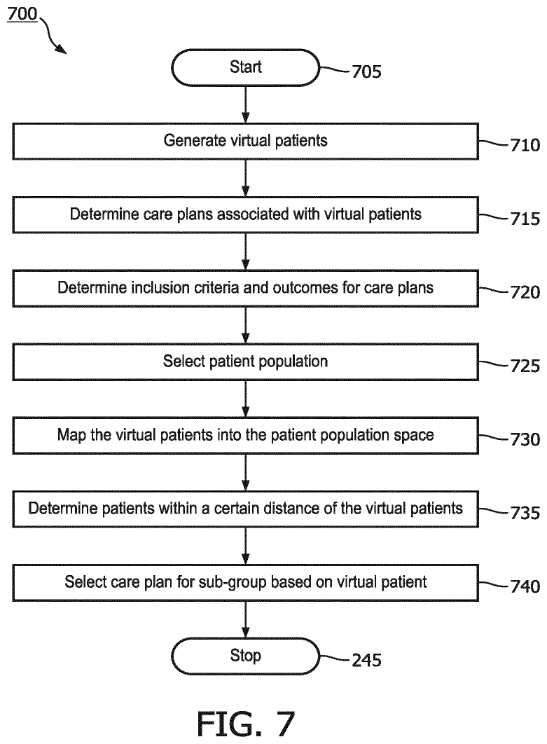

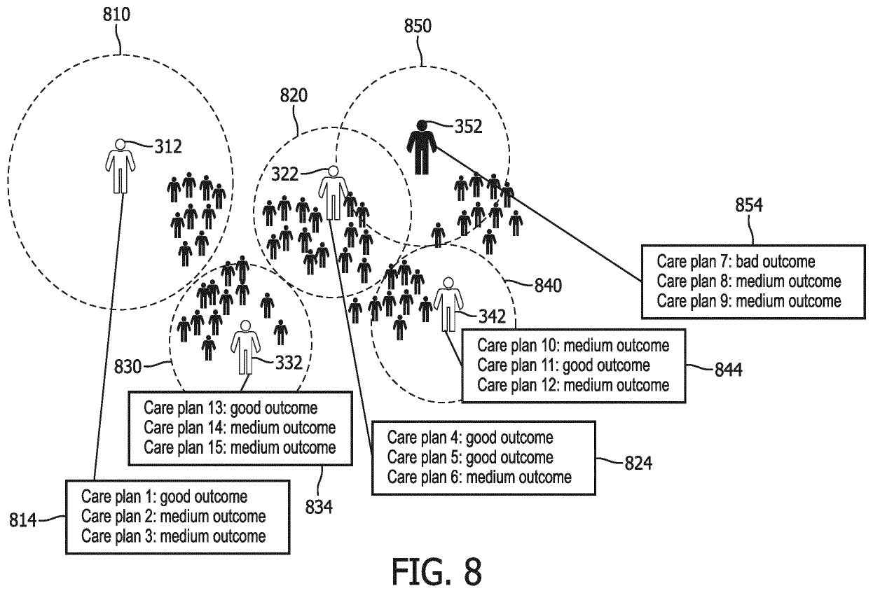

FIG. 7 illustrates third a care plan assignment method 700. FIG. 8 provides an illustration of the application of the care plan assignment method 700 of FIG. 7 to a patient population. The care plan assignment method 700, for each unseen patient, selects the most similar virtual patient based upon the following steps. The care plan assignment method 700 starts at 705 and then generates virtual patients 710 as described above using the method 100. The care plan assignment method 700 then determines the care plans associated with the virtual patients 715 as well as the inclusion criteria and outcomes for the care plans 720 as shown in FIG. 3. The care plan assignment method 700 then selects a patient population 725. This may be accomplished via input from a user of the system or automatically performed. Then the care plan assignment method 700 maps the virtual patients into the patient population space 730. This is shown in FIG. 8 where the virtual patients 312, 322, 332, 342, and 352 are mapped among the patient population. The care plan assignment method 700 then determines the patients within a certain distance from each of the virtual patients 735. Defining this certain distance may be done in various ways including using the boundaries and distances of the sub-groups from the clustering of the patients used to define the virtual patients in step 115. This is shown in FIG. 8 by the groupings 814, 824, 834, 844, and 854 around each virtual patient. Then the care plan assignment method 700 selects a care plan for each sub-group based on the associated virtual patient 740. This is shown in FIG. 8 where the list of care plans 814, 824, 834, 844, and 854 associated with the virtual patients 312, 322, 332, 342, and 352 are shown. The best care plan in the list of care plans will be selected for each patient in the sub-group subject to inclusion and eligibility criteria for the care plans. As a result, patients in the same sub-group may have different care plans assigned because of inclusion and eligibility criteria. The method then ends at 745. An effect of this care plan assignment method 700 is that the active patients are not grouped together based on similarity but based on similarity to the virtual patient. Another effect is that some patients may be positioned in an overlap area between two groups as seen for groups 820 and 850. In such a situation patients may be assigned to the closest virtual patient based on the smallest distance of this patient to the respective virtual patient.

For each of the care plan assignment methods, a measure of confidence of the patient-care plan matching may be derived by comparing patients in a sub-group to the virtual patient whose care plan assignment is suggested to the patient based upon characteristics that are important to the care plan (e.g., the characteristics used in the inclusion/exclusion criteria, outcomes, etc.). By determining the distance between a patient to the virtual patient and comparing against a threshold (or against other distances observed within the cluster), small distances may be given a high confidence level and those with larger distances a lower level of confidence. These confidence levels may be provided to a care provider using the care plan assignment method. Further, the proposed care plan assignment may be displayed to the care provider with the option to make corrections and acknowledge the plan by the care provider.

The data processing system solves the technological problem of matching a specific subject to desired outcome associated with another subject or group of subjects in a large subject population. The computation for matching a specific subject with one of a large number of subjects becomes very computationally expensive. The data processing system uses clustering techniques to identify a smaller number of virtual subjects that are representative of the subject population as a whole. Comparing specific subjects to this much smaller set of virtual subjects results in a large decrease in the computation cost. This allows for such comparisons to be made in a timelier fashion and for a larger number of subjects when computational resources are limited.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method for generating a care plan for each patient of a patient population, comprising:
collecting patient data including features for a plurality of patients;
clustering the patient data based upon the features to define patient data sub-groups;
determining the homogeneity of the patient data sub-groups;
generating virtual patients for each patient data sub-group that represent the features of the patient data sub-group;
determining care plans associated with each virtual patient;
selecting a patient population;
and performing a first process, a second process or a third process,
wherein the first process comprises:
adding the virtual patients to the patient population;
clustering the patient population with the virtual patients to define patient sub-groups in the patient population;
identifying the virtual patient or virtual patients in each patient sub-group; and
selecting a care plan for each patient in the patient sub-group based upon the virtual patient or virtual patients in the patient sub-group
wherein the second process comprises:
clustering the patient population to define patient sub-groups in the patient population;
adding the virtual patients to their nearest respective patient sub-group of the patient population; and
selecting a care plan for each patient in the patient sub-groups based upon the virtual patient or virtual patients associated with the patient sub-groups, and
wherein the third process comprises:
mapping the virtual patients into the patient population space;
determining which patients are within a certain distance of each virtual patient and
selecting a care plan for each patient based upon the virtual patient which is within the certain distance of the patient.

2. The method of claim 1, wherein generating the virtual patient includes selecting an actual patient based upon the mode of the patient data for the patient data sub-group.

3. The method of claim 1, wherein generating the virtual patient includes defining the features of the virtual patient based upon the average of the patient data for the patient data sub-group.

4. The method of claim 1, wherein generating the virtual patient includes defining the features of the virtual patient based upon the median of the patient data for the patient data sub-group.

5. The method of claim 1, further comprising clustering a patient data sub-group when the homogeneity of said patient data sub-group is below a specified value.

6. The method of claim 1, wherein the first process is performed.

7. The method of claim 6, wherein selecting a care plan for each patient in the patient sub-group is further based upon one of patient inclusion criteria and patient eligibility criteria.

8. The method of claim 7, further comprising determining the inclusion criteria for each care plan associated with each virtual patient.

9. The method of claim 1, wherein the second process is performed.

10. The method of claim 9, wherein selecting a care plan for each patient in the patient sub-group is further based upon one of patient inclusion criteria and patient eligibility criteria.

11. The method of claim 10, further comprising determining the inclusion criteria for each care plan associated with each virtual patient.

12. The method of claim 1, wherein the third process is performed.

13. The method of claim 12, wherein selecting a care plan for each patient is further 6based upon one of patient inclusion criteria and patient eligibility criteria.

14. The method of claim 13, further comprising determining the inclusion criteria for each care plan associated with each virtual patient.

15. The method of claim 13, wherein selecting a care plan for each patient further includes, when a patient is within the certain distance of two virtual patients, selecting the care plan associated with the virtual patient closest to the patient.

16. A non-transitory machine-readable storage medium encoded with instructions for generating a care plan for each patient of a patient population, comprising:
instructions for collecting patient data including features for a plurality of patients;
instructions for clustering the patient data based upon the features to define patient data sub-groups;
instructions for determining the homogeneity of the patient data sub-groups;
instructions for generating virtual patients for each patient data sub-group that represent the features of the patient data sub-group;
instructions for determining care plans associated with each virtual patient;
instructions for selecting a patient population; and
a first set of instructions, a second set of instructions or a third set of instructions,
wherein the first set of instructions comprises
instructions for adding the virtual patients to the patient population;
instructions for clustering the patient population with the virtual patients to define patient sub-groups in the patient population;
instructions for identifying the virtual patient or virtual patients in each patient sub-group; and
instructions for selecting a care plan for each patient in the patient sub-group based upon the virtual patient or virtual patients in the patient sub-group,
wherein the second set of instructions comprises:
instructions for clustering the patient population to define patient sub-groups in the patient population;
instructions for adding the virtual patients to the nearest patient sub-group of the patient population; and
instructions for selecting a care plan for each patient in the patient sub-groups based upon the virtual patient or virtual patients associated with the patient sub-group,
wherein the third set of instructions comprises:
instructions for mapping the virtual patients into the patient population space;
instructions for determining which patients are within a certain distance of each virtual patient; and
instructions for selecting a care plan for each patient based upon the virtual patient within the certain distance of the patient.

17. The non-transitory machine-readable storage medium of claim 16, wherein instructions for generating the virtual patient includes instructions for selecting an actual patient based upon the mode of the patient data for the patient data sub-group.

18. The non-transitory machine-readable storage medium of claim 16, wherein instructions for generating the virtual patient includes instructions for defining the features of the virtual patient based upon the average of the patient data for the patient data sub-group.

19. The non-transitory machine-readable storage medium of claim 16, wherein instructions for generating the virtual patient includes instructions for defining the features of the virtual patient based upon the median of the patient data for the patient data sub-group.

20. The non-transitory machine-readable storage medium of claim 16, further comprising instructions for clustering a patient data sub-group when the homogeneity of said patient data sub-group is below a specified value.

21. The non-transitory machine-readable storage medium of claim 16, comprising the first set of instructions.

22. The non-transitory machine-readable storage medium of claim 21, wherein instructions for selecting a care plan for each patient in the patient sub-group is further based upon patient one of patient inclusion criteria and patient eligibility criteria.

23. The non-transitory machine-readable storage medium of claim 22, further comprising instructions for determining the inclusion criteria for each care plan associated with each virtual patient.

24. The non-transitory machine-readable storage medium of claim 16, comprising the second set of instructions.

25. The non-transitory machine-readable storage medium of claim 24, wherein instructions for selecting a care plan for each patient in the patient sub-group is further based upon one of patient inclusion criteria and patient eligibility criteria.

26. The non-transitory machine-readable storage medium of claim 25, further comprising instructions for determining the inclusion criteria for each care plan associated with each virtual patient.

27. The non-transitory machine-readable storage medium of claim 16, comprising the third set of instructions.

28. The non-transitory machine-readable storage medium of claim 27, wherein instructions for selecting a care plan for each patient is further based upon one of patient inclusion criteria and patient eligibility criteria.

29. The non-transitory machine-readable storage medium of claim 28, further comprising instructions for determining the inclusion criteria for each care plan associated with each virtual patient.

30. The non-transitory machine-readable storage medium of claim 29, wherein instructions for selecting a care plan for each patient further includes, when a patient is within the certain distance of two virtual patients, instructions for selecting the care plan associated with the virtual patient closest to the patient.
